SEQUENCE LISTING (36 sequences)

SEQ ID NO: 1 - DNA, 20 bp, Artificial Sequence
  Synthetic Gapdh_forward
gacgtgcagcaggaacacta

SEQ ID NO: 2 - DNA, 20 bp, Artificial Sequence
  Synthetic Gapdh_reverse
cttggactttgccagagagg

SEQ ID NO: 3 - DNA, 20 bp, Artificial Sequence
  Synthetic Il-1-Beta_forward
gcatcaagggctacaagctc

SEQ ID NO: 4 - DNA, 20 bp, Artificial Sequence
  Synthetic Il-1-Beta_reverse
caggcggtagaagatgaagc

SEQ ID NO: 5 - DNA, 20 bp, Artificial Sequence
  Synthetic Il81_forward
cctcctgcctcctacattca

SEQ ID NO: 6 - DNA, 20 bp, Artificial Sequence
  Synthetic Il81_reverse
atctccagctcctttcacga

SEQ ID NO: 7 - DNA, 20 bp, Artificial Sequence
  Synthetic Il-17a_forward
gaactgccttgcctaacagc

SEQ ID NO: 8 - DNA, 20 bp, Artificial Sequence
  Synthetic Il-17a_reverse
tcttctcatggagcacgttg

SEQ ID NO: 9 - DNA, 20 bp, Artificial Sequence
  Synthetic Tnf-alpha_forward
agatgggaagggaatgaacc

SEQ ID NO: 10 - DNA, 20 bp, Artificial Sequence
  Synthetic Tnf-alpha_reverse
gacgtgtcacgatcatctgg

SEQ ID NO: 11 - DNA, 20 bp, Artificial Sequence
  Synthetic Ripk3_forward
cttgggatttaccgctacca

SEQ ID NO: 12 - DNA, 20 bp, Artificial Sequence
  Synthetic Ripk3_reverse
agcaggaaggaggctgtgta

SEQ ID NO: 13 - DNA, 20 bp, Artificial Sequence
  Synthetic Ptgs2_forward
accagcatttcaacctttgc

SEQ ID NO: 14 - DNA, 20 bp, Artificial Sequence
  Synthetic Ptgs2_reverse
ccaggttgctgctctactcc

SEQ ID NO: 15 - DNA, 20 bp, Artificial Sequence
  Synthetic Slc25a4_forward
aagcgctcagctttctcaag

SEQ ID NO: 16 - DNA, 20 bp, Artificial Sequence
  Synthetic Slc25a4_reverse
tctccgctgtgatctgtttg

SEQ ID NO: 17 - DNA, 21 bp, Artificial Sequence
  Synthetic CjRsmE_forward
tccaccaaagcactcaagaat

SEQ ID NO: 18 - DNA, 24 bp, Artificial Sequence
  Synthetic CjRsmE_reverse
attgttaaatccaccttcaggacc

SEQ ID NO: 19 - DNA, 20 bp, Artificial Sequence
  Synthetic Cp16S_forward
caacttgggtgctgcattcc

SEQ ID NO: 20 - DNA, 20 bp, Artificial Sequence
  Synthetic Cp16S _reverse
gcctcagcgtcagttacagt

SEQ ID NO: 21 - DNA, 20 bp, Artificial Sequence
  Synthetic Emax18S_forward
gacctcggtcaccgtatcac

SEQ ID NO: 22 - DNA, 20 bp, Artificial Sequence
  Synthetic Emax18S_reverse
cgtgcagcccagaacatcta

SEQ ID NO: 23 - DNA, 20 bp, Artificial Sequence
  Synthetic Bact16S_forward
craacaggattagataccct

SEQ ID NO: 24 - DNA, 19 bp, Artificial Sequence
  Synthetic Bact16S_reverse
ggtaaggttcctcgcgtat

SEQ ID NO: 25 - DNA, 21 bp, Artificial Sequence
  Synthetic Firm16S_forward
tgaaactyaaaggaattgacg

SEQ ID NO: 26 - DNA, 17 bp, Artificial Sequence
  Synthetic Firm16S_reverse
accatgcaccacctgtc

SEQ ID NO: 27 - DNA, 19 bp, Artificial Sequence
  Synthetic alpha-Prote16S_forward
  misc_feature (2)..(2): n is inosine
cnagtgtagaggtgaaatt

SEQ ID NO: 28 - DNA, 21 bp, Artificial Sequence
  Synthetic alpha-Prote16S _reverse
ccccgtcaattcctttgagtt

SEQ ID NO: 29 - DNA, 19 bp, Artificial Sequence
  Synthetic Gamma-Prote16S_forward
tcgtcagctcgtgtygtga

SEQ ID NO: 30 - DNA, 16 bp, Artificial Sequence
  Synthetic Gamma-Prote16S_reverse
cgtaagggccatgatg

SEQ ID NO: 31 - DNA, 16 bp, Artificial Sequence
  Synthetic Actin16S_forward
tacggccgcaaggcta

SEQ ID NO: 32 - DNA, 19 bp, Artificial Sequence
  Synthetic Actin16S_reverse
tcrtccccaccttcctccg

SEQ ID NO: 33 - DNA, 20 bp, Artificial Sequence
  Synthetic Univ16S_forward
ctcctacggggaggcagcaa

SEQ ID NO: 34 - DNA, 15 bp, Artificial Sequence
  Synthetic Univ16S_reverse
acgggcggtgtgtrc

SEQ ID NO: 35 - DNA, 20 bp, Artificial Sequence
  Synthetic Cj16S_forward
cagctcgtgtcgtgagatgt

SEQ ID NO: 36 - DNA, 20 bp, Artificial Sequence
  Synthetic Cj16S_reverse
gcataagggccatgatgact

(19) United States
(12) Patent Application Publication
(10) Pub. No.: US 2018/0360072 A1
(43) Pub. Date: Dec. 20, 2018

(54) BILE ACID COMPOSITIONS AND METHODS OF USE

(71) Applicant: THE BOARD OF TRUSTEES OF THE UNIVERSITY OF ARKANSAS, Little Rock, AR (US)

(72) Inventor: Xiaolun Sun, Fayetteville, AR (US)

(73) Assignee: THE BOARD OF TRUSTEES OF THE UNIVERSITY OF ARKANSAS, Little Rock, AR (US)

(21) Appl. No.: 16/009,398

(22) Filed: Jun. 15, 2018

Related U.S. Application Data
(60) Provisional application No. 62/520,724, filed on Jun. 16, 2017.
(60) Provisional application No. 62/529,569, filed on Jul. 7, 2017.

(51) Int. Cl. A23K 20/168; A23K 20/147; A61K 31/575; A61K 47/42; A61K 47/26; A61P 31/04; A61P 33/00

(52) U.S. Cl. CPC A23K 20/168; A23K 20/147; A61K 31/575; A61P 33/00; A61K 47/26; A61P 31/04; A61K 47/42

(57) ABSTRACT

Animal feed compositions including a bile acid or a salt thereof are provided. Also provided are bile acid modulated intestinal digesta compositions. The present invention further relates to methods of using such compositions to improve body weight gain or the accumulative feed conversion ratio in an agricultural animal species and to prevent and/or reduce the growth of a pathogenic bacteria or a parasite in the intestinal tract of an agricultural animal species.

### CROSS-REFERENCE TO RELATED PATENT APPLICATIONS

The present application claims the benefit of priority to U.S. Provisional Patent Application No. 62/520,724, filed on Jun. 16, 2017, and U.S. Provisional Patent Application No. 62/529,569, filed on Jul. 7, 2017, the contents of which are incorporated herein by reference in their entireties.

### SEQUENCE LISTING

This application is being filed electronically via EFS-Web and includes an electronically submitted Sequence Listing in .txt format. The .txt file contains a sequence listing entitled “2018-06-15_5658-00427_ST25.txt” created on Jun. 14, 2018 and is 7,562 bytes in size. The Sequence Listing contained in this .txt file is part of the specification and is hereby incorporated by reference herein in its entirety.

### INTRODUCTION

Antimicrobial resistance is an emerging challenge to both agriculture and healthcare (Geisinger and Isberg, 2017; Khan et al., 2017; Watkins and Bonomo, 2016). Limiting antimicrobial usage in agriculture is one of the important steps to control antimicrobial resistance. However, antimicrobial free feeding regiments practiced around the world have precipitated outbreak of intestinal diseases (coccidiosis, necrotic enteritis (NE)), productivity loss (Gaucher et al., 2015) and increased food borne pathogens such as Campylobacter (Pham et al., 2016; Wise and Siragusa, 2007). It is, therefore, an urgent need to find effective alternatives to antimicrobial feeding regiments.

Campylobacter jejuni is a member of normal poultry microbiota but a prevalent human food borne bacterial pathogen. Campylobacteriosis cases are estimated at millions every year, and ranked second among all food borne pathogens (CDC, 2014, 2018), but with limited prevention and treatment options and occasional severe post-infection complications (Boyanova et al., 2004; Gradel et al., 2009; Newman and Lambert, 1980; Speed et al., 1984).

Few effective approaches except limited antimicrobials are currently available to reduce C. jejuni colonization and necrotic enteritis (NE) outbreaks in agricultural animals such as chickens. Recent research advancement on microbiome, however, brings new hopes for sustainably resolving C. jejuni colonization and NE. Notably, human recurrent Clostridium difficile enteric infection has been successfully treated with stool from healthy donor when antibiotic treatment failed (Silverman et al., 2010). We found that specific pathogen free Il110 mouse resistance against C. jejuni colonization was lost when the mice were treated with antibiotic clindamycin. The antibiotic treatment altered microbiota and microbial metabolites of bile acids which are metabolized by specific groups of microbes. Furthermore, secondary bile acid deoxycholate (DCA) inhibited host response of mTOR signaling pathway and treated C. jejuni-induced intestinal inflammation in germ free Il10^{-/-} mice.

Bile acids are synthesized from cholesterol in the liver. The two primary bile acids synthesized in the human liver are cholic acid (CA), and chenodeoxycholic acid (CDCA) (Mataki et al., 2007). Bile acids are further metabolized by the liver via conjugation to glycine or taurine, a modification that protects hosts. The gut microbiota impacts bile acid metabolism by promoting deconjugation, dehydrogenation, and dehydroxylation of primary and secondary bile acids in the small intestine, colon and cecum, thus transforming bile acids (Ridlon et al., 2006). High concentrations of bile salts are maintained in the duodenum, jejunum, and proximal ileum. Most bile acids (>95%) are absorbed and reused, a process called enterohepatic circulation. Some secondary bile acids inhibit human pathogen Clostridium difficile growth in vitro (Buffie et al., 2015). Whether the microbial metabolites of the bile acids resist growth of enteric pathogens at the organism level, such as in poultry, is unclear.

### SUMMARY

In one aspect, the present invention relates to bile acid modulated intestinal digesta. Bile acid modulated intestinal digesta may be made by a method including administering a bile acid or a salt thereof to an agricultural animal species and isolating an intestinal digesta from the agricultural animal species. Optionally, the intestinal digesta may be further cultured under either aerobic or anaerobic conditions to make bile acid modulated aerobe intestinal digesta and bile acid modulated anaerobe intestinal digesta, respectively.

In another aspect of the present invention, animal feed compositions are provided. The animal feed compositions may include a bile acid or salt thereof or any one of the bile acid modulated intestinal digesta described herein and a protein component, carbohydrate component, or both a protein component and a carbohydrate component.

In another aspect, the present invention relates to methods for improving body weight gain or the accumulative feed conversion ratio in an agricultural animal species. The methods may include administering to the agricultural animal species an effective amount of a composition including a bile acid or a salt thereof or any one of the bile acid modulated intestinal digesta described herein.

In a further aspect, the present invention relates to methods for preventing and/or reducing the growth of a pathogenic bacteria or a parasite in the intestinal tract of an agricultural animal species. The methods may include administering to the agricultural animal species an effective amount of a composition comprising a bile acid or a salt thereof or any one of the bile acid modulated intestinal digesta described herein.

### BRIEF DESCRIPTION OF THE DRAWINGS

FIG. 1 shows DCA resists against C. jejuni colonization. Cohorts of 13 broiler chicks were fed basal and DCA diets. The birds were orally gavaged with 10⁹ CFU/chick C. jejuni 81-176 at 14 days of age. Birds were sacrificed at 0 (14 days of age), 2, 7 and 14 days C. jejuni post infection (PI). The cecal content was serially diluted and microaerobically cultured on selective C. jejuni plates.

FIG. 2 shows DCA promotes broiler growth performance. Cohorts of 13 broiler chicks were fed basal and DCA diets. Growth performance of body weight gain and feed intake was recorded at day 7, 14, 18 and 28. Feed conversion ratio was calculated and lower value means better feed efficiency. (FIG. 2A) Accumulative body weight gain. (FIG. 2B) Accumulative feed conversion ratio.

FIG. 3 shows DCA modifies microbiota. Cohorts of 13 broiler chicks were fed basal and DCA diets. Cecal content from birds at 28 days of age was collected and subjected to DNA isolation. Real time PCR was performed to measure microbiota using phylum level specific primers. The results were expressed as percentage to total microbiota of five phyla. Actinobacteria, α-proteobacteria, γ-proteobacteria were insignificant percentages of the total microbiota measured.

FIG. 4 shows DCA-modulated microbiota reduces C. jejuni chicken colonization. Birds colonized with DCA-modulated anaerobe (DCA-Anaero) and aerobe (DCA-Aero) were infected with C. jejuni AR 101 at d 10 and the bacterium in ceca was cultured on d 28.

FIGS. 5A-5B show DCA-modulated anaerobes attenuate exacerbated chicken-transmitted campylobacteriosis. SPF Il10^{-/-} mice were gavaged with clindamycin for 7 days and then infected with 10⁹ CFU/mouse C. jejuni Cj-P0, Cj-P1, and Cj-P1-DCA-Anaero. The mice were sacrificed after 8 days of infection. (FIG. 5A) Representative H&E images of colitis. (FIG. 5B) Histopathological score. *, P<0.05.

FIGS. 6A-6B show DCA doesn't alter fat and nitrogen digestion and absorption. Cohorts of 10 day old chicks were fed 0 and 1.5 g/kg DCA diet and growth performance was recorded. (FIG. 6A) Daily Body weight gain of birds at 26 days of age. (FIG. 6B) Fat and nitrogen levels at ileal content of 8 birds at 26 days of age. All graphs depict mean±SEM. **, P<0.01; NS, not significant.

FIG. 7 shows DCA modulates host inflammatory response. Cohorts of 10 day old chicks were fed 0 and 1.5 g/kg DCA diet as in FIG. 4. After upper ileal tissues were collected, RNA was extracted. Ptgs2 and Atp6vg1 mRNA accumulation was quantified using a Bio-Rad PCR System and specific primers and data were normalized to Gapdh. All graphs depict mean±SEM. *, P<0.05.

FIGS. 8A-8B show DCA inhibits C. perfringens in vitro growth. Equivalent dose of C. perfringens was inoculated in TSB broth supplemented with 0.05% thioglycollate. Various concentrations of bile acids were added in the broth. The bacterial growth was recorded and measured using OD600. (FIG. 8A) Image of C. perfringens growth with different bile acids. The cloud broth shows growth well and the clear broth indicates growth inhibition. (FIG. 8B) Bacterial growth measured using OD600.

FIGS. 9A-9B show DCA attenuates coccidiosis- and necrotic enteritis-induced productivity loss. Cohorts of 13 day old chicks were fed basal diet, 1.5 g/kg CA or 1.5 g/kg DCA diets and growth performance of body weight gain and feed efficiency were measured. To induce necrotic enteritis (NE), the birds were infected with 20,000 oocysts/bird E. maxima (Em) at 18 days of age and then infected with 10⁹ CFU/bird C. perfringens at 23 days of age. The birds were sacrificed on 26 days of age. (FIG. 9A) Periodic daily body weight gain. (FIG. 9B) Accumulative feed conversion ratio.

FIG. 10A-10B shows DCA attenuates E. maxima- and C. perfringens-induced necrotic enteritis. Cohorts of 13 day old chicks were fed basal diet, 1.5 g/kg CA or 1.5 g/kg DCA diets and induced necrotic enteritis (NE) with E. maxima (Em) and C. perfringens infection as in FIG. 9. FIG. 10A is H&E staining showing representative intestinal histopathology of NE-induced intestinal inflammation of coccidiosis and NE in broiler chickens at 26 days of age. FIG. 10B shows quantification of histological intestinal damage score. All graphs depict mean±SEM. *, P<0.05; **, P<0.01. Scale bar is 200 μm.

FIG. 11 shows DCA attenuates NE-induced inflammatory response. Cohorts of 13 day old chicks were fed basal diet, 1.5 g/kg CA or 1.5 g/kg DCA diets and infected as in FIG. 9. After upper ileal tissues were collected, RNA was extracted. Infγ, Tnfα and Mmp9 mRNA accumulation was quantified using a Bio-Rad PCR System and specific primers and data were normalized to Gapdh. All graphs depict mean±SEM. *, P<0.05.

FIG. 12 shows NE and ileal luminal C. perfringens. Cohorts of 13 day old chicks were fed basal diet, 1.5 g/kg CA or 1.5 g/kg DCA diets and infected as in FIG. 9. C. perfringens at ileal luminal was quantified using 16s rDNA qPCR. All graphs depict mean±SEM. NS, not significant. Results are representative of 2 independent experiments.

FIG. 13 shows DCA attenuates C. perfringens virulence. Cohorts of 13 day old chicks were fed basal diet, 1.5 g/kg CA or 1.5 g/kg DCA diets and infected as in FIG. 9. After upper ileal tissues were collected, RNA was extracted. C. perfringens virulence gene 16s, Cpb and Virx mRNA accumulation was quantified using a Bio-Rad PCR System and specific primers and data were normalized to Gapdh. All graphs depict mean±SEM. *, P<0.05.

FIG. 14 shows DCA reduces E. maxima activity. Cohorts of 13 day old chicks were fed basal diet, 1.5 g/kg CA or 1.5 g/kg DCA diets and infected as in FIG. 9. After upper ileal tissues were collected, RNA was extracted. E. maxima 18S mRNA accumulation was quantified and normalized to Gapdh. All graphs depict mean±SEM. *, P<0.05; **, P<0.01.

### DETAILED DESCRIPTION

Antimicrobial resistance is an emerging challenge to both agriculture and healthcare. Limiting antimicrobial usage in agriculture is one of the important steps to control antimicrobial resistance. However, antimicrobial free feeding regiments practiced around the world have precipitated the outbreak of intestinal diseases (e.g. necrotic enteritis) and food borne diseases (e.g. campylobacteriosis). For example, Campylobacter jejuni is a poultry commensal microorganism but a food borne bacterial pathogen in humans with increasing antibiotic resistance incidences. Cases of campylobacteriosis ranked second among all food borne pathogens with limited treatment options and severe post-infection complications.

In the non-limiting Examples, the present inventors demonstrate that bile acid-modulating microbiota and host response prevents against C. jejuni transmission in chicken and resists against necrotic enteritis (NE). The inventors further demonstrate that bile acids such as deoxycholic acid (DCA) and DCA modulated intestinal digesta promote broiler growth performance, prevented C. jejuni colonization, and modulated microbiota and host inflammation responses. Additionally, bile acids reduced C. perfringens in vitro growth and attenuated Eimeria Maxima and C. perfringens-induced NE. Furthermore, dietary primary bile acid CA reduces bird NE intestinal inflammation and NE-induced growth performance loss (e.g. body weight gain and feed efficiency). In the face of increasing need for antimicrobial free feeding regimens and safe food, the identification of microbial metabolites reducing C. jejuni and NE represents a significant step forward in developing effective strategies to reduce food borne pathogens and to improve agricultural animal productivity.

The present inventors contemplate that bile acids and/or bile acid modulated intestinal digesta delivered in animal feed, drinking water, or other routes may improve animal productivity and prevent prevalent food-borne bacterial pathogens such as Campylobacter jejuni colonization in chickens. The bile acids or digesta may also reduce coccidiosis- and Clostridium perfringens-induced necrotic enteritis and attenuate other animal intestinal pathogens. Altogether this invention provides a novel, effective and sustainable approach for antimicrobial free feeding regimens in agricultural animal production and animal health.

In one aspect, the present invention relates to bile acid modulated intestinal digesta. Bile acid modulated intestinal digesta may be made by a method including administering a bile acid or a salt thereof to an agricultural animal species and isolating an intestinal digesta from the agricultural animal species. Optionally, the intestinal digesta may be further cultured under either aerobic or anaerobic conditions to make bile acid modulated aerobe intestinal digesta and bile acid modulated anaerobe intestinal digesta, respectively.

Suitably, the intestinal digesta may be obtained from any part of an intestine including, without limitation, the cecum, duodenum, jeunum, or ileum.

In another aspect of the present invention, animal feed compositions are provided. The animal feed compositions may include a bile acid (or salt thereof) and/or any one of the bile acid modulated intestinal digesta described herein and a protein component, carbohydrate component, or both a protein component and a carbohydrate component. Optionally, the animal feed compositions may further include a fat component, a mineral component, a vitamin component, or any combination thereof.

As used herein, a “bile acid” refers to steroid acids found in the bile of vertebrates. The bile acid may be either a primary bile acid or a secondary bile acid.

As used herein, a “primary bile acid” refers to those bile acids synthesized from cholesterol in the liver. Primary bile acids may include, without limitation, cholic acid (CA) and chenodeoxycholic acid (CDCA).

As used herein, a “secondary bile acid” refers to compounds that are commonly formed by intestinal bacteria from primary bile acids. Exemplary secondary bile acids may include, without limitation, deoxycholic acid, lithocholic acid, ursodeoxycholic acid, or common derivatives thereof. Suitably, the secondary bile acid is deoxycholic acid (DCA). Common derivatives of DCA may include HDCA, hyodeoxycholic acid (3α,6α-dihydroxy-5β-cholanoic acid); LCA, lithocholic acid (3α-hydroxy-5β-cholanoic acid); MDCA, murideoxycholic acid (3α,6β-dihydroxy-5β-cholanoic acid); and UDCA, ursodeoxycholic acid (3α,7β-dihydroxy-5β-cholanoic acid).

The animal feed compositions may include key nutrients needed to meet the dietary requirements of a particular animal. These key nutrients may include a protein component, a carbohydrate component, fats and oils, minerals, vitamins, or any combination thereof.

Common protein components found in animal feeds may include, without limitation, protein meals or protein derived from vegetable and animal sources, such as soybean, oilseed meals, legumes, abattoir, and fish processing by-products. Suitable oilseed meals may include, without limitation, soybean, rapeseed/canola, sunflower, palm kernel, copra, linseed peanut sesame seed meals.

Common carbohydrate components found in animal feeds may include, without limitation, cereal grains such as corn, wheat, sorghum, barley, rye, triticale, or oats.

In some embodiments, the animal feed composition may be a poultry feed composition. As used herein, a “poultry feed composition” is poultry feed commonly used to raise poultry. Exemplary poultry feed compositions are well-described in the art and may be found, for example, in Poultry Nutrition 5th Edition, written by W. Ray Ewing The Ray Ewing Co. Pasadena Calif. 1963.

The bile acid or salt thereof may be within the animal feed composition at a concentration ranging from 0.01-10 g/kg feed, 0.1-5 g/kg feed, 0.5-2 g/kg feed, or any range therein.

Bile acid modulated intestinal digesta, including bile acid modulated aerobe intestinal digesta and bile acid modulated anaerobe intestinal digesta, may be within the animal feed composition at a concentration ranging from 10⁴-10¹² CFU per dose, 10⁶-10¹⁰ per dose or any range therein.

In another aspect, the present invention relates to methods for improving body weight gain or the accumulative feed conversion ratio in an agricultural animal species. The methods may include administering to the agricultural animal species an effective amount of a composition including a bile acid or a salt thereof or any one of the bile acid modulated intestinal digesta described herein.

As used herein, “administering” may be carried out through any of the variety of procedures used to apply compositions to agricultural animal species that will be apparent to the skilled artisan. Suitable application methods may include incorporating the bile acid or salt thereof or bile acid modulated intestinal digesta into the feed or water source for the agricultural animal. Accordingly, the compositions used in accordance with the present methods may be any one of the animal feed compositions disclosed herein. In some embodiments, the composition of the present methods may be a liquid including water such as drinking water that may be given to the agricultural animal species.

As used herein, an “agricultural animal species” may include any domesticated animal species used to produce agricultural products. Suitable agricultural animal species may include, without limitation, a poultry species, a cow species, a pig species, a sheep species, a goat species, or a fish species. Preferably, the agricultural animal species is a poultry species such as a chicken or turkey species. In some embodiments, the agricultural animal species are suffering from, or is at risk of developing, campylobacteriosis, necrotic enteritis or other infectious enteritis. In some embodiments, the agricultural animal species are transmitting food borne pathogens such as C. jejuni and C. perfringens.

In accordance with the present methods, the body weight gain of the agricultural animal species may be improved by increasing by at least 5%, 10%, 20%, 30%, 40%, or 50% the body weight of the agricultural animal as compared to a control animal.

As used herein, a “control animal” refers to an agricultural animal species that is given a composition that excludes the bile acid or salt thereof or a bile acid modulated intestinal digesta. For example, an appropriate control animal for a chicken species that is subjected to the present methods would be the same chicken species feed a composition that excludes the bile acid or salt thereof or a bile acid modulated intestinal digesta.

In accordance with the present methods, the accumulative feed conversion ratio of the agricultural animal species may be improved by decreasing the accumulative feed conversion ratio by at least 5%, 10%, 20%, 30%, 40%, or 50% as compared to a control animal.

In a further aspect, the present invention relates to methods for preventing and/or reducing the growth of a pathogenic bacteria or a parasite in the intestinal tract of an agricultural animal species. The methods may include administering to the agricultural animal species an effective amount of a composition comprising a bile acid or a salt thereof or any one of the bile acid modulated intestinal digesta described herein.

The “pathogenic bacteria or parasite” may be any bacteria or parasite that adversely affects the health of the agricultural animal species or the health of a species that may consume the agricultural animal species/products from the agricultural animal species (i.e., humans). Suitable pathogenic bacteria or parasites may include, without limitation, Campylobacter jejuni, Eimeria maxima, or Clostridium perfringens.

The growth of the pathogenic bacteria or the parasite in the intestinal tract of an agricultural animal species may be reduced by at least 30%, 40%, 50%, 55%, 65%, 70%, 80% or 100% as compared to a control animal. In some embodiments, the reduced growth of the pathogenic bacteria or the parasite may result in improved body weight gain or an accumulative feed conversion ratio as described herein.

The term “effective amount” is intended to mean an amount of a composition described herein sufficient to improve body weight gain or the accumulative feed conversion ratio in an agricultural animal species; or to prevent and/or reduce the growth of a pathogenic bacteria or a parasite in the intestinal tract of an agricultural animal species. When the bile acid or salt thereof is administered in an animal feed composition, the bile acid or salt thereof may be found in the animal feed composition in a concentration ranging from 0.01-10 g/kg feed, 0.1-5 g/kg feed, 0.5-2 g/kg feed, or any range therein. When the bile acid or salt thereof is administered in the drinking water of the agricultural animal species, the secondary bile acid or salt thereof may be found in the water composition in a concentration ranging from 0.01-10 g/L water, 0.1-5 g/L water feed, 0.5-2 g/L water, or any range therein.

When the bile acid modulated intestinal digesta is administered, the digesta may be administered at 10⁴-10¹² CFU per dose, 10⁴-10⁸ CFU per dose, 10⁶-10¹⁰ per dose, or any range within these ranges.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1—Bile Acids Against Food Animal Borne Intestinal Pathogens

### Materials and Methods

### Chicken Experiments

All animal protocols were approved by the Institutional Animal Care and Use Committee of the University of Arkansas. A cohort of day-old broiler chicks (13 chicks/pen) was transferred from a commercial hatchery to a poultry isolator room at John Kirkpatrick Skeeles Poultry Health Laboratory (up to Biosafety level 3) at University of Arkansas at Fayetteville. The birds were fed standard starter diet from 0 to 10 days of age and then fed grower diets from 11 to 28 days of age. Birds also fed 1.5 g/kg cholic acid (CA) or deoxycholic acid (DCA) diets.

The birds were infected with human clinical isolator C. jejuni strain 81-176 at 10⁹ colony form unit (CFU)/bird at 14 days of age. Bird weight and feed intake were measured at 7, 14, 18 and 28 days of age. Birds were sacrificed at 0 (14 days of age), 2, 7 and 14 days C. jejuni post infection (PI). The experiment was terminated at 14 days C. jejuni PI. Intestinal tissue and content were collected for molecular analysis and C. jejuni culture enumeration. The cecal content was serially diluted and microaerobically cultured on selective C. jejuni plates supplemented with 5 antibiotics cocktail (cefoperazone, cycloheximide, trimethoprim, vancomycin and polymyxin B). In our laboratory, these five-antibiotic cocktail plate effectively identified C. jejuni from non-specific antibiotic-resistant bacterial colonies. Tissue samples were assayed for inflammatory response on mRNA accumulation of Ripk3, Ptgs2, Slc25a4, Il1β, Tnfα, Il8, Cxcl2, and Il17α. DNA from intestinal content was extracted and selected bacterial population at phylum or species levels was assessed by real time PCR.

In a separate experiment, birds were fed diet supplementing with DCA at 1.5 g/kg feed. After 26 days the birds were weighed and sacrificed and ileal content were collected. The dry matter (DM), crude fat, and nitrogen levels in the ileal content was analyzed at the Central Analytical Lab at Department of Poultry Science at University of Arkansas at Fayetteville, Ark.

Extracting DNA and RNA from Intestinal Content and Tissue

Bird cecal content was weighed and DNA was extracted using bead beater disruption and phenol:chloroform separation. Briefly, intestinal content was combined with beads, PBS, and phenol:chloroform. After bead beater disruption and centrifugation, supernatant was extracted once with phenol:chloroform and once with chloroform. DNA in the supernatant was precipitated using ethanol and sodium acetate method. After centrifugation, the pellet was washed using 70% ethanol twice. The final DNA pellet was dissolved in DNase/RNase free water and stored in −80° C. freezer.

Total RNA from chicken intestinal tissue was extracted using TRIzol (Invitrogen, Carlsbad, Calif.). Briefly, after bead beater disruption in TRIzol and chloroform and centrifugation, supernatant was precipitated with isopropanol. The pelleted RNA was washed once with 75% ethanol. The final RNA was dissolved in DNase/RNase free water and stored in −80° C. freezer. Complementary DNA was prepared using M-MLV (NEB).

### Real-Time Reverse Transcription Polymerase Chain Reaction

Messenger RNA levels of proinflammatory genes were determined using SYBR Green PCR Master Mix (Bio-Rad, Hercules, Calif.) on an Bio-Rad 384-well Real-Time PCR System and normalized to Gapdh. The polymerase chain reactions were performed on 384-well real time PCR machine (Bio-Rad, Hercules, Calif.) according to the manufacturer's recommendation. The following gene primers were used: Gapdh_forward: 5′-gacgtgcagcaggaacacta-3′ (SEQ ID NO: 1), Gapdh_reverse: 5′-cttggactttgccagagagg-3′ (SEQ ID NO: 2), Il-1β_forward: 5′-GCATCAAGGGCTACAAGCTC-3′ (SEQ ID NO: 3), Il-1β_reverse: 5′-CAGGCGGTAGAAGATGAAGC-3′ (SEQ ID NO: 4), Il81_forward: 5′-cctcctgcctcctacattca-3′ (SEQ ID NO: 5), Il81_reverse: 5′-atctccagctcctttcacga-3′ (SEQ ID NO: 6), Il-7a_forward: 5′-gaactgccttgcctaacagc-3′ (SEQ ID NO: 7), Il-17a_reverse: 5′-tcttctcatggagcacgttg-3′ (SEQ ID NO: 8); Tnfα_forward: 5′-agatgggaagggaatgaacc-3′ (SEQ ID NO: 9), Tnfα_reverse: 5′-gacgtgtcacgatcatctgg-3′ (SEQ ID NO: 10); Ripk3_forward 5′-cttgggatttaccgctacca-3′ (SEQ ID NO: 11), Ripk3_reverse 5′-agcaggaaggaggctgtgta-3′ (SEQ ID NO: 12); Ptgs2_forward 5′-accagcatttcaacctttgc-3′ (SEQ ID NO: 13), Ptgs2_reverse 5′-ccaggttgctgctctactcc-3′ (SEQ ID NO: 14), Slc25a4_forward 5′-aagcgctcagctttctcaag-3′ (SEQ ID NO: 15), Slc25a4_reverse 5′-tctccgctgtgatctgtttg-3′ (SEQ ID NO: 16).

Bacteria in the intestinal content was determined using the Real Time PCR. The DNA was diluted 100 time to reduce inhibition of PCR reaction from the intestinal content. The following gene primers were used: CjRsmE_forward: 5′-TCCACCAAAGCACTCAAGAAT-3′ (SEQ ID NO: 17), CjRsmE_reverse: 5′-ATTGTTAAATCCACCTTCAGGACC-3′, (SEQ ID NO: 18) Cp16S_forward: 5′-CAACTTGGGTGCTGCATTCC-3′ (SEQ ID NO: 19), Cp16S_reverse: 5′-GCCTCAGCGTCAGTTACAGT-3′ (SEQ ID NO: 20), Emax18S_forward: 5′-GACCTCGGTCACCGTATCAC-3′ (SEQ ID NO: 21), Emax18S_reverse: 5′-CGTGCAGCCCAGAACATCTA-3′ (SEQ ID NO: 22); Bact16S_forward: 5′-CRAACAGGATTAGATACCCT-3′ (SEQ ID NO: 23), Bact16S_reverse: 5′-GGTAAGGTTCCTCGCGTAT-3′ (SEQ ID NO: 24); Firm16S_forward: 5′-TGAAACTYAAAGGAATTGACG-3′ (SEQ ID NO: 25), Firm16S_reverse: 5′-ACCATGCACCACCTGTC-3′ (SEQ ID NO: 26); αProte16S_forward: 5′-CIAGTGTAGAGGTGAAATT-3′ (SEQ ID NO: 27), αProte16S_reverse: 5′-CCCCGTCAATTCCTTTGAGTT-3′ (SEQ ID NO: 28); γProte16S_forward: 5′-TCGTCAGCTCGTGTYGTGA-3′ (SEQ ID NO: 29), γProte16S_reverse: 5′-CGTAAGGGCCATGATG-3′ (SEQ ID NO: 30); Actin16S_forward: 5′-TACGGCCGCAAGGCTA-3′ (SEQ ID NO: 31), Actin16S_reverse: 5′-TCRTCCCCACCTTCCTCCG-3′ (SEQ ID NO: 32); Univ16S_forward: 5′-CTCCTACGGGGAGGCAGCAA-3′ (SEQ ID NO: 33), Univ16S_reverse: 5′-ACGGGCGGTGTGTRC-3′ (SEQ ID NO: 34); Cj16S_forward: 5′-cagctcgtgtcgtgagatgt-3′ (SEQ ID NO: 35), Cj16S_reverse: 5′-gcataagggccatgatgact-3′ (SEQ ID NO: 36). The microbiota PCR data were calculated as percentage relative to combined phylum bacteria.

C. jejuni In Vitro Growth Inhibition

To evaluate bile acids on C. jejuni growth, the bacterium was inoculated in BHI broth. The medium was also added final concentration of 0, 0.2, 0.5, and 1 mM DCA, and 1 mM TCA and CA. After 48-hour culture under microaerobic condition, the bacterium growth was measured at OD₆₀₀.

### Chicken Microbiota Collection and Transplantation

Cecal digesta from birds fed DCA diet were collected at 28 days of age. The digesta were immediately suspended in 30% glycerol PBS stock and stored at −80° C. as DCA microbiota (DCA-Biota). DCA-Biota was then cultured under aerobic or anaerobic conditions for 48 hours on Brain Hart Infusion (BHI) agar plates and collected as DCA aerobes (DCA-Aero) and DCA anaerobes (DCA-Anaero), respectively.

To colonize chickens with microbiota, one-day-old birds were orally gavaged with 108 CFU/bird DCA-Anaero or DCA-Aero. After ten-day colonization, the birds were infected with C. jejuni chicken isolate AR101 (Cj-P0), which was isolated at Dr. Billy Hargis's lab at University of Arkansas at Fayetteville. The birds were sacrificed at 28 days of age and cecal C. jejuni colonization were determined on select plates. C. jejuni was then isolated from infected birds as Cj-P1 or from birds infected and colonized with DCA-Anaero as Cj-P1-DCA-Anaero.

Chicken-Transmitted C. jejuni Induced Intestinal Inflammation in Il10−/− Mice Specific pathogen free (SPF) Il10−/− mice at 8-12 weeks of age were gavaged with clindamycin at 67 mg/kg body weight (BW) for 7 days to deplete secondary bile acid metabolizing bacteria. After 24 hours of washing period, the mice were infected with Cj-P0. Cj-P1, or Cj-P1-DCA-Anaero. The mice were monitored daily for clinic signs of illness and sacrificed at 8 days post infection. Colon tissue was collected for H&E staining and histopathological score were evaluated as describe before (Lippert et al., 2009).

### Statistical Analysis

Values are shown as mean±standard error of the mean as indicated. Differences between groups were analyzed using the t-test. Experiments were considered statistically significant if P values were <0.05. Calculations were performed using Prism 5.0 software.

### Results:

DCA Improves Growth Performance and Prevents C. jejuni Colonization

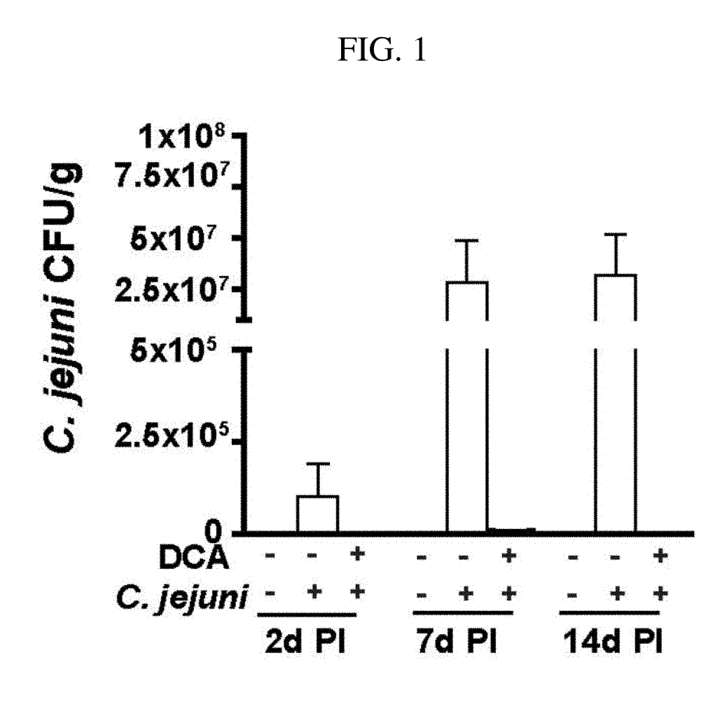

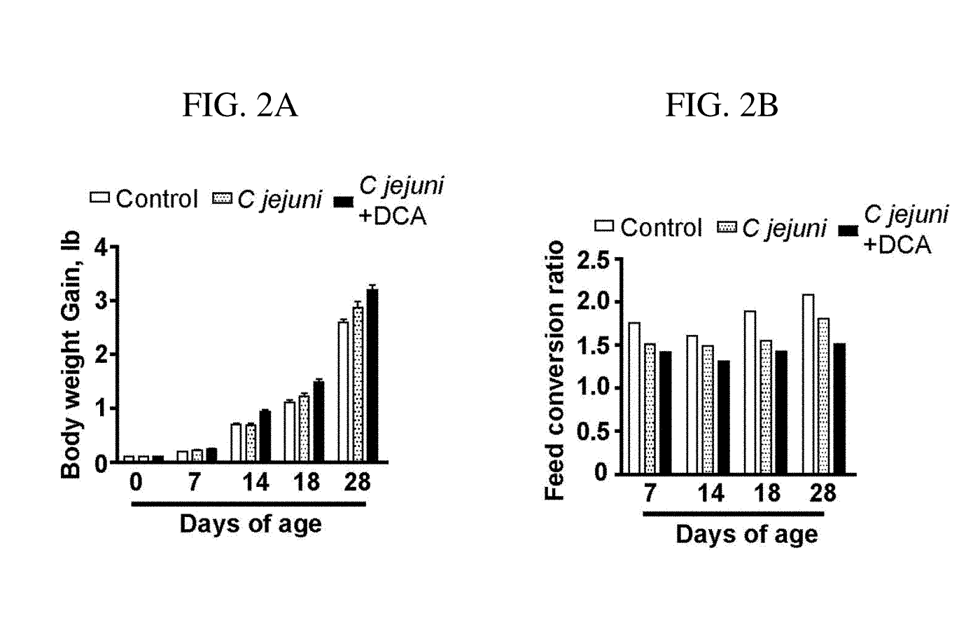

To evaluate the role of bile acids on C. jejuni colonization in chickens, broiler chicks at 14 days of age was gavaged with human clinical C. jejuni isolate 81-176 at 10⁹ CFU/chick. Interestingly, C. jejuni quickly colonized in bird's ceca at 1.0×10⁵ CFU/g cacal content at 2 days post infection (PI) and reached to 2.8×10⁷ CFU/g and 3.1×10⁷ CFU/g at 7 and 14 days PI, respectively (FIG. 1). Importantly, broiler chickens fed 1.5 g/kg DCA diet dramatically resisted against C. jejuni colonization at levels of 0, 9.0×10², and 0 CFU/g at 2, 7 and 14 days PI, respectively. Furthermore, DCA diet improved broilers productivity of body weight gain at 3.20 vs. 2.86 lb at 28 days of age, 11.9% increase (FIG. 2A) and accumulative feed conversion ratio at 1.51 vs. 1.81, 16.6% improvement (FIG. 2B) compared to control diet.

### DCA Modulates Chicken Cecal Microbiota

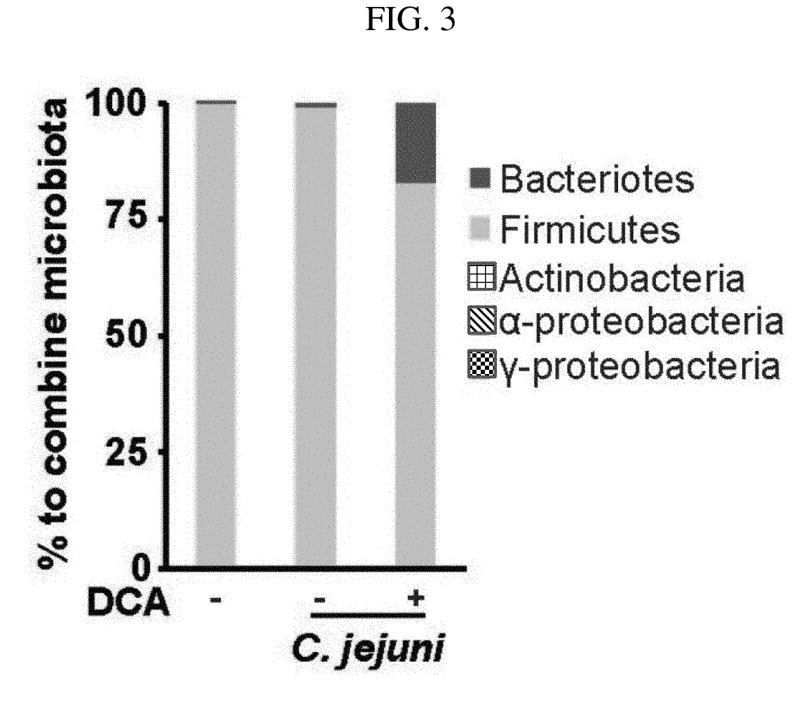

To elucidate the underlying mechanism of DCA on promoting chicken productivity, we then collected chicken cecal content and analyzed microbiota composition using phylum-specific real time PCR. C. jejuni infection/colonization didn't impact cecal microbiota distribution (FIG. 3). Interestingly, DCA diet modified microbiota at ceca, where phylum bacteroidetes was increased (16.94 vs. 0.06%) but firmicutes was decreased (82.71 vs. 99.58%) compared to control diet.

DCA-Modulated Anaerobes Reduce C. jejuni Chicken Colonization

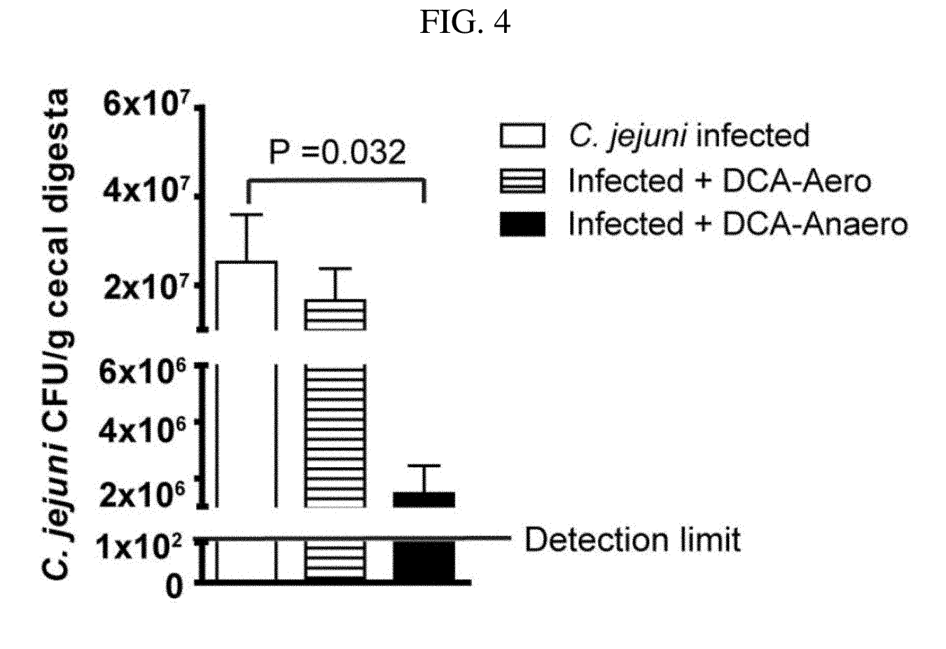

Since DCA reduced C. jejuni chicken colonization (FIG. 1) and changed bird cecal microbiota at phylum level (FIG. 3), we hypothesized that DCA-modulated microbiota might contribute to C. jejuni colonization reduction. To investigate this possibility, we cultured cecal microbiota from DCA-fed birds under aerobic and anaerobic condition and collected them as DCA-Aero and DCA-Anaero, respectively. Day old broiler chicks were inoculated with 10⁸ CFU/bird DCA-Aero or DCA-Anaero. The birds were infected with 10⁹ CFU/bird chicken isolate C. jejuni AR 101 at 10 days of age. AR 101 was kindly provided by Dr. Billy Hargis. Cecal digesta was collected and cultured on selective plates 28 days of age. No C. jejuni were detected in birds without infection. Consistent with previous observations, C. jejuni AR 101colonized in chicken ceca at level of 2.5×10⁷ CFU/g cecal digesta at 28 days of age (FIG. 4). Notably, DCA-Anaero reduced C. jejuni cecal colonization by 94.1%, while DCA-Aero failed to reduce the bacterial colonization in ceca. These data suggest that DCA reduces C. jejuni chicken colonization, possibly through modulating cecal anaerobes, but not aerobes.

### DCA-Modulated Anaerobes Attenuate Chicken Transmission-Exacerbated Campylobacteriosis Using Il10^{-/-} Mouse Infection Models

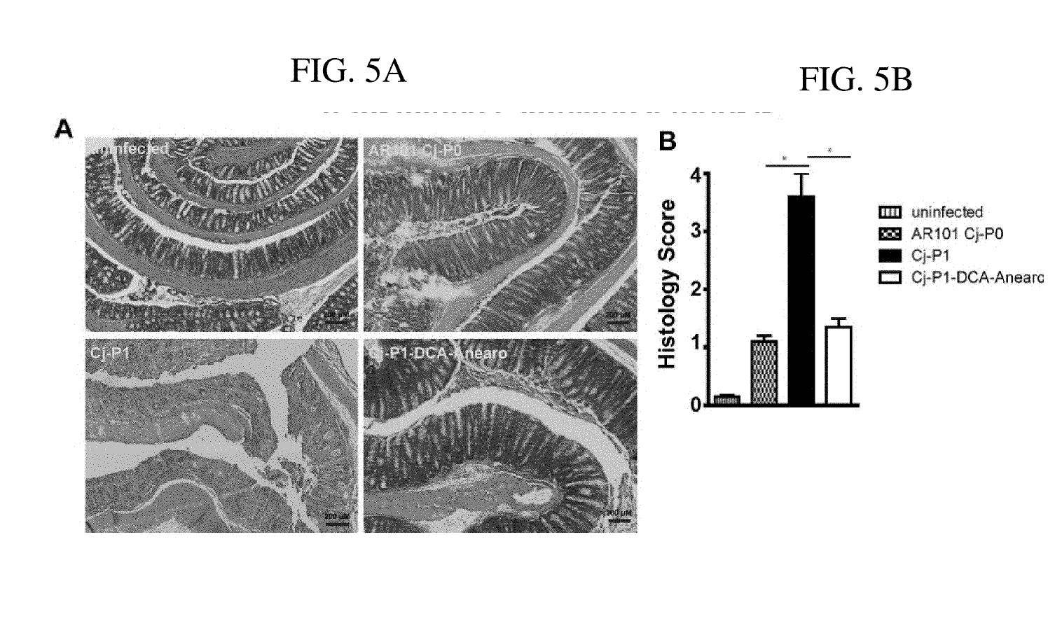

Traditionally, food bacterial pathogen contamination is determined by enumerating bacterial pathogens by culture or PCR. However, this approach overlooks the possibility of bacterial virulence alteration after the bacterial colonization in food. To address this important issue in C. jejuni chicken transmission, we collected C. jejuni AR 101 from infected alone (C. jejuni passage 1, Cj-P1) and infected and DCA-Anearo colonized birds (Cj-P1-DCA-Anaero). Specific pathogen free (SPF) Il10^{-/-} mice were orally gavaged for 7 days with clindamycin to deplete secondary bile acid metabolizing bacteria as describe before (Sun et al., 2018). 24 hours after last clindamycin administration, the mice were then infected with C. jejuni AR 101 (Cj-P0), Cj-P1, or Cj-P1-DCA-Anaero. The mice were sacrificed 8 days after C. jejuni infection. Consistent with previous reports (Sun et al., 2018), C. jejuni Cj-P0 induced intestinal inflammation and increased histopathology score in Il10^{-/-} mice, showed as crypt hyperplasia and immune cell infiltration (FIGS. 5A and 5B). Remarkably, chickens-transmitted C. jejuni Cj-P1 induced more severe campylobacteriosis compared to non-transmitted Cj-P0, suggesting increased virulence of C. jejuni after its chicken colonization and transmission. Importantly, DCA-modulated anaerobes attenuated the exacerbated camylobacteriosis compared to Cj-P1. These data indicate that C. jejuni increases virulence and induction of campylobacteriosis after chicken transmission, and specific microbiota could attenuate the increased virulence and infectious enteritis.

DCA Doesn't Alter C. jejuni Growth and Fat and Protein Digestion and Absorption

To address if DCA directly inhibit C. jejuni growth, we cultured C. jejuni in brain heart infusion (BHI) broth supplementing with various concentrations of DCA for 48 hours. Surprisingly, physiological level DCA at 1 mM slightly enhanced but not inhibit C. jejuni growth for 2 days in BHI broth (OD₆₀₀ at 0.213 vs. 0.153), suggesting that DCA may not directly target the pathogen growth. Interestingly, 1 mM TCA or CA also failed to inhibit C. jejuni growth (OD₆₀₀ at 0.197 and 0.19, respectively).

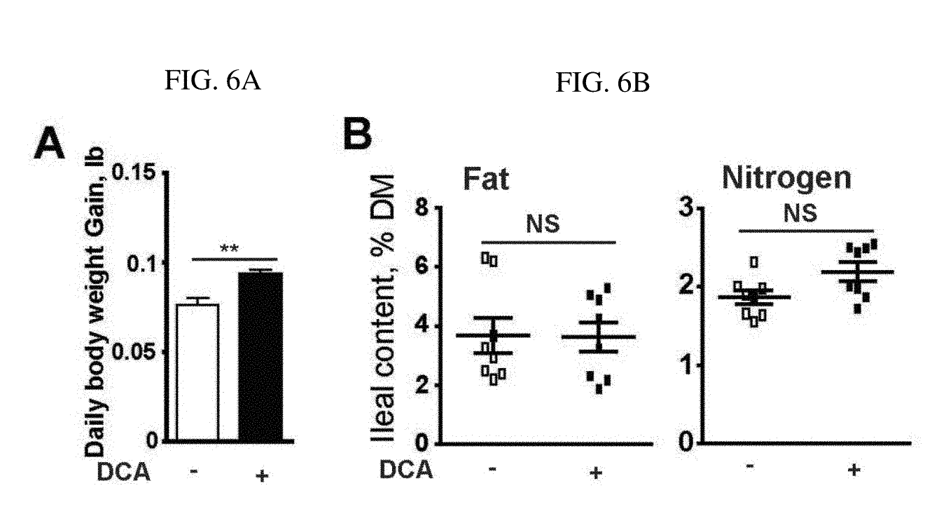

Because bile acids are important mediators of dietary fat digestion and absorption, we then conducted another growth experiment, in which birds were fed basal diet and 1.5 g/kg feed DCA diet for 26 days and weighed body weight and feed intake. We also collected bird ileal content and measured nitrogen and fat levels. Consistent with previous experiment, DCA diet promoted bird growth performance on body weight gain (FIG. 6A), but interestingly, fat and nitrogen concentrations in ileal content between DCA and basal diet birds were comparable (FIG. 6B). These results suggest that the effects of DCA eliminating chicken C. jejuni colonization and promoting growth are independent on bacterial niche exclusion or on digestion and absorption of fat and protein.

### DCA Mediates Host Response

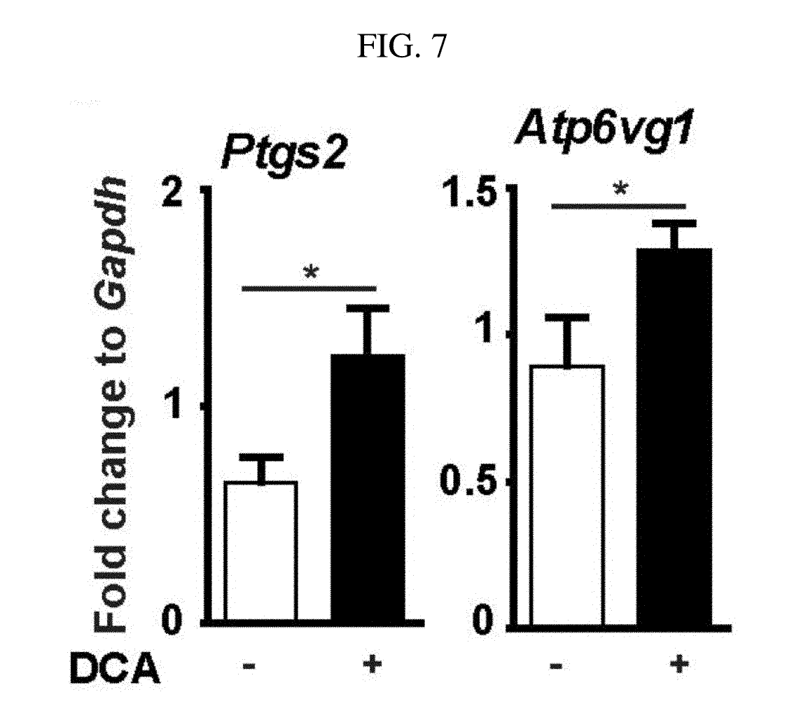

We then reasoned that DCA may mediate host responses. Ileal tissue were collected and RNA was extracted for reverse transcription and real time PCR. Among many gene expressions screened, DCA diet modulated host response gene mRNA accumulation of Ptgs2 (+92%) and Atp6vg1 (+40%) in chicken intestinal tissue compared to control diet (FIG. 7). Altogether, these results indicate that DCA improving bird growth performance and excluding C. jejuni colonization may depend on mechanisms of modulating host response and microbiota, but not from altering intestinal digestion and absorption of dietary fat and protein or not from inhibit the bacterial growth.

### Example 2—Bile Acids Against Animal Intestinal Pathogens

### Materials and Methods

### Chicken Experiments

A cohort of day-old broiler chicks (13 chicks/pen) was transferred from a commercial hatchery to a poultry isolator room at John Kirkpatrick Skeeles Poultry Health Laboratory (up to Biosafety level 3) at University of Arkansas at Fayetteville. The birds were fed standard starter diet from d 0 to 10 days of age and then fed grower diets from d 11 to 27 days of age. Birds also fed 1.5 g/kg cholic acid or deoxycholic acid (DCA) diets.

Birds were orally infected with Eimeria maxima at 20,000 oocysts/bird at 18 days of age. The birds were then infected with C. perfringens at 10⁹ CFU/bird at 23 days of age to induce NE. Bird weight and feed intake were measured at 7, 14, 18, 23, 26, and 27 days of age. Birds were sacrificed at 0 (23 days of age), 3 and 4 days C. perfringens post infection (PI). The experiment was terminated at 4 days C. perfringens PI. Intestinal tissue and content were collected for histology and molecular analysis. Tissue samples were assayed for inflammatory response on mRNA accumulation of Infγ, Ripk3, Ptgs2, Slc25a4, Il1β, Tnfα, Il81, Mmp9, Cxcl2, and Il17a. DNA from intestinal content was extracted and selected bacterial population at phylum or species levels was assessed by real time PCR. Histopathology images were acquired using a DP71 camera and DP Controller 3.1.1.276 (Olympus). Ileal pathology was evaluated and scored.

Extracting DNA and RNA from Intestinal Content and Tissue

Bird upper ileal content was weighed and DNA was extracted using bead beater disruption and phenol:chloroform separation. Briefly, intestinal content was combined with beads, PBS, and phenol:chloroform. After bead beater disruption and centrifugation, supernatant was extracted once with phenol:chloroform and once with chloroform. DNA in the supernatant was precipitated using ethanol and sodium acetate method. After centrifugation, the pellet was washed using 70% ethanol twice. The final DNA pellet was dissolved in DNase/RNase free water and stored in −80° C. freezer.

Total RNA from chicken intestinal tissue was extracted using TRIzol (Invitrogen, Carlsbad, Calif.). Briefly, after bead beater disruption in TRIzol and chloroform and centrifugation, supernatant was precipitated with isopropanol. The pelleted RNA was washed once with 75% ethanol. The final RNA was dissolved in DNase/RNase free water and stored in −80° C. freezer. Complementary DNA was prepared using M-MLV (NEB).

### Real-Time Reverse Transcription Polymerase Chain Reaction

Messenger RNA levels of proinflammatory genes were determined using SYBR Green PCR Master Mix (Bio-Rad, Hercules, Calif.) on an Bio-Rad 384-well Real-Time PCR System and normalized to Gapdh. The polymerase chain reactions were performed on 384-well real time PCR machine (Bio-Rad, Hercules, Calif.) according to the manufacturer's recommendation. The gene primers were used as showed in Example 1. Bacteria in the intestinal content was determined using the Real Time PCR. The DNA was diluted 100 time to reduce inhibition of PCR reaction from the intestinal content.

C. perfringens In Vitro Growth Inhibition

To assess the impact of bile acids on C. perfringens growth, the bacterium was inoculated in Tryptic Soy Broth (TSB) plus thioglycollate. The medium was also added final concentration of 0, 0.2, 0.4, 0.6, 0.8, 1, and 2 mM DCA, 0.6 and 1 mM TCA and CA. After overnight culture under anaerobic condition, the bacterium growth was recorded for OD₆₀₀ reading.

### Statistical Analysis

Values are shown as mean±standard error of the mean as indicated. Differences between groups were analyzed using the t-test. Experiments were considered statistically significant if P values were <0.05. Calculations were performed using Prism 5.0 software.

### Results

DCA Inhibits C. perfringens In Vitro Growth

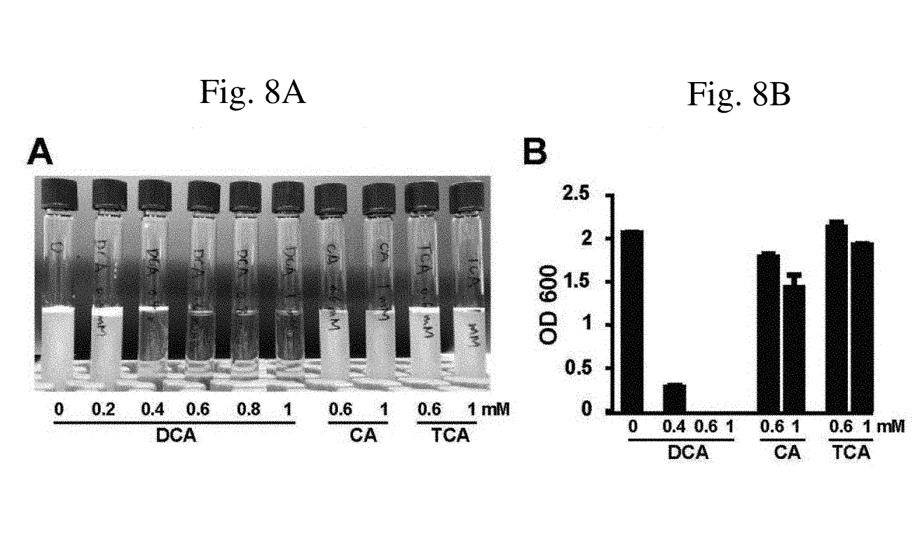

To address whether DCA attenuates coccidia E. maxima- and C. perfringens-induced necrotic enteritis (NE), we first ran an in vitro inhibition experiment, in which C. perfringens was inoculated in Tryptic Soy Broth (TSB) with various concentrations of conjugated primary bile acid taurocholic acid (TCA), primary bile acid cholic acid (CA) and secondary bile acid DCA. Interestingly, DCA inhibited 87% C. perfringens growth at 0.4 mM (clear broth) while TCA at as high as 1 mM failed to prevent its growth (cloudy broth) and CA at 1 mM only reduced 30% bacterial growth (FIGS. 8A and 8B).

DCA Attenuates Coccidiosis and C. perfringens-Induced Necrotic Enteritis and Productivity Loss

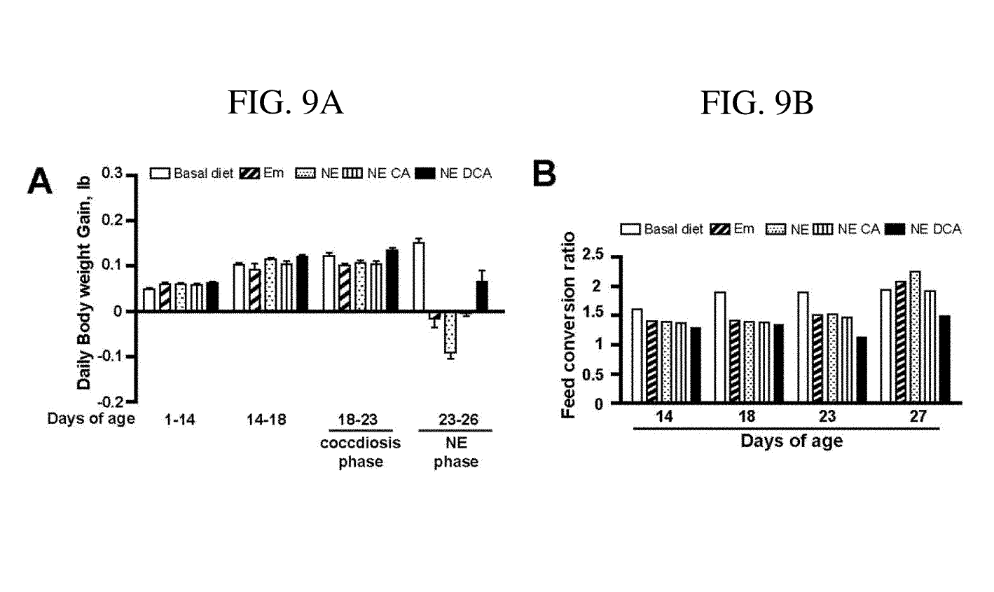

To further investigate whether DCA reduces NE in birds, we fed day old broiler chicks with 1.5 g/kg CA and 1.5 g/kg DCA diets and growth performance of body weight gain and feed efficiency were measured. To induce NE, the birds were infected with 20,000 oocysts/bird E. maxima at 18 days of age and then infected with 10⁹ CFU/bird C. perfringens at 23 days of age. Remarkably, DCA diet promoted bird growth performance on body weight gain (FIG. 9A) and feed efficiency (FIG. 9B) during 1-14 and 14-18 days of age compared to birds fed other diets. Significant impairment on growth performance of body weight gain and feed efficiency were observed at coccidiosis phase during 18-23 days of age and at NE phase during 23-26 days of age in Em and NE birds. Notably, DCA prevented productivity loss at both coccidiosis phase and NE phase compared to Em and NE control birds. Primary bile acid CA diet attenuated body weight gain loss at NE phase (d 23-26) but failed at coccidiosis phase (d 18-23) compared to NE control birds.

### DCA Alleviates Coccidiosis and NE-Induced Intestinal Inflammation

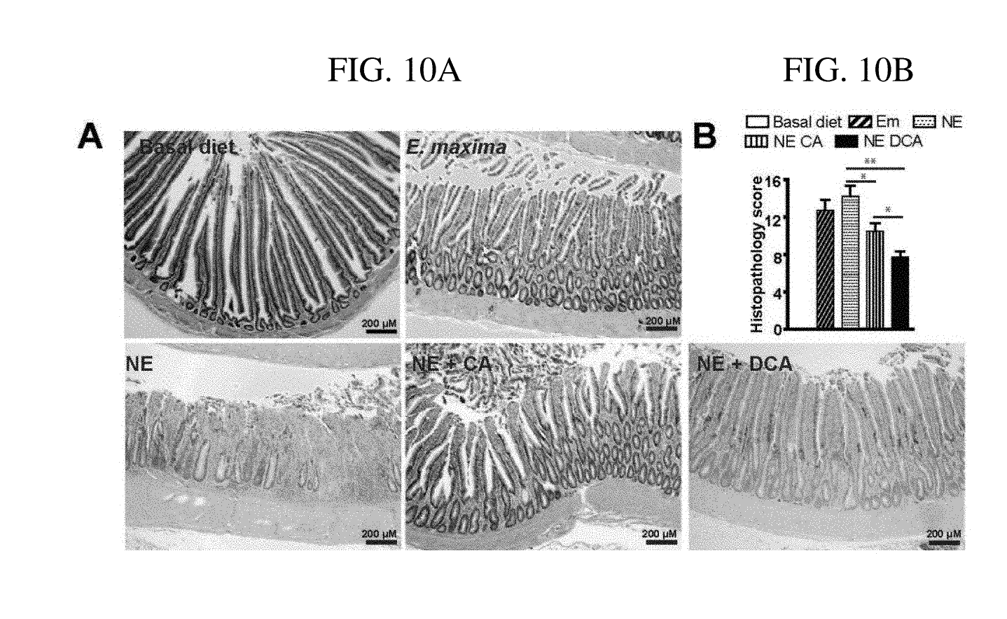

To have thorough insight of NE pathogenesis, we collected intestinal tissue at upper ileum as Swiss rolls, processed with H&E staining, and performed histopathology analysis. Notably, E. maxima infection induced severe intestinal inflammation as seen by immune cell infiltration into lamina propria, crypt hyperplasia and mild villus height shortening compared to uninfected birds (FIG. 10A). Furthermore, NE control birds endured worse intestinal inflammation as seen by necrosis of villi and crypt, massive immune cell infiltration and severe villus shortening. Remarkably, DCA diet significantly attenuated NE-induced intestinal inflammation and histopathology score (FIGS. 10A and 10B). These results indicate that microbial metabolite DCA promotes growth performance and resists against coccidiosis- and NE-induced productivity loss.

### DCA Ameliorates NE-Induced Inflammatory Response

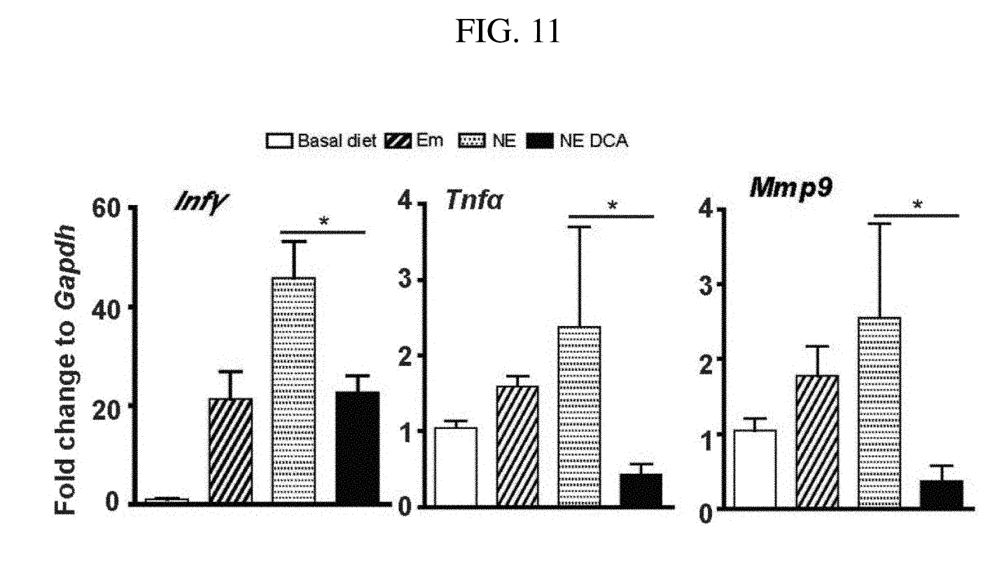

Because DCA reduced NE-induced intestinal inflammation, we collected ileal tissue and examined various inflammatory mediator gene expression using real time PCR. Consistent with increased intestinal inflammation, Em induced inflammatory gene expression of Infγ, Tnfα and Mmp9 compared to uninfected birds (FIG. 11). Notably, NE strongly induced Infγ, Tnfα and Mmp9 mRNA accumulation in ileal tissue of NE control birds, effects attenuated by 51%, 82%, and 66% in NE birds fed DCA diet, respectively (FIG. 11).

DCA Mollifies C. perfringens and E. maxima Virulence

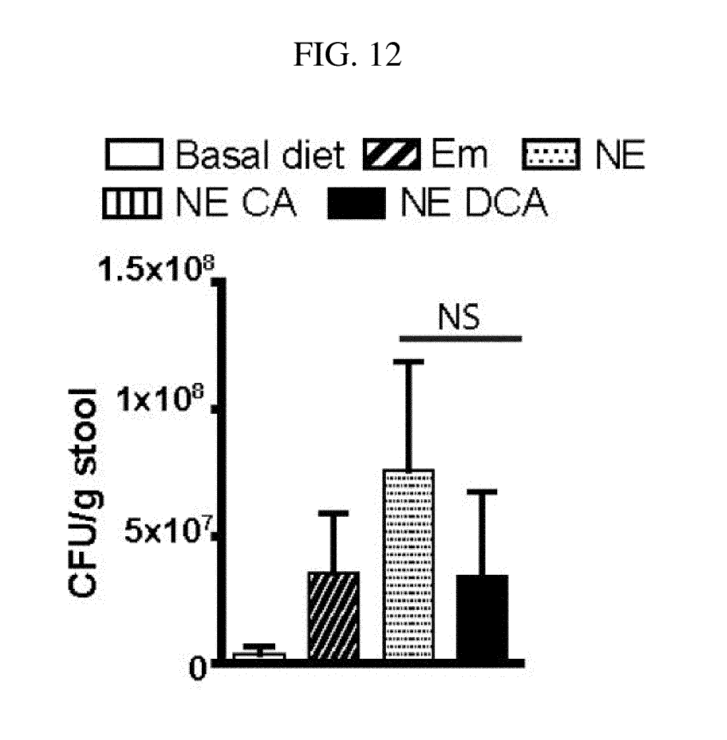

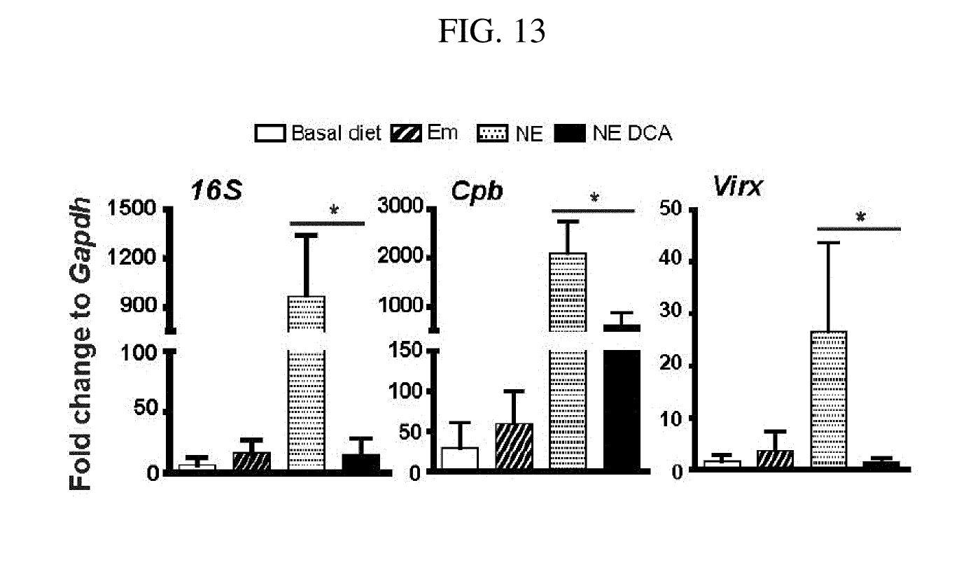

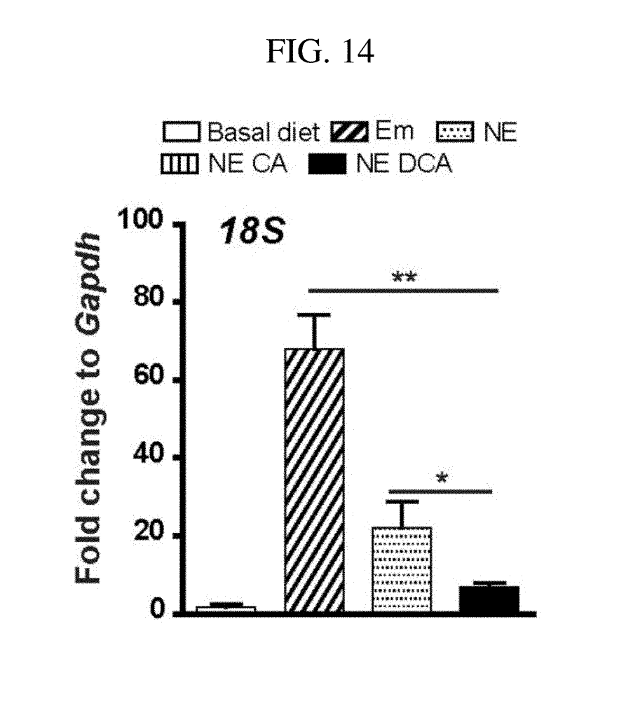

To examine if the pathogen overgrowth in intestine played a role in NE, we collected ileal content, extracted total DNA, and measured C. perfringens colonization level in intestinal lumen using real time PCR of 16S rDNA. As showed in FIG. 10A-10B, coccidiosis and NE were associated with elevated C. perfringens compared to uninfected birds. Surprisingly, ileal luminal C. perfringens load in DCA birds was not significantly different from NE control birds (FIG. 12), while bird productivity and histopathology were strikingly distinct between the two groups of birds. We therefore reasoned that the pathogen invasion into tissue and its activity was the main driving factors of NE pathogenesis, but not the pathogen luminal colonization level. We then measured the various C. perfringens virulence gene expression in ileal tissue. NE was associated with strong induction of the bacterial 16S, Cpb and Virx mRNA accumulation in ileal tissue of NE control birds and DCA blocked them by 97%, 61%, and 61%, respectively (FIG. 13). We also examined E. maxima activity by measuring its 18S gene expression. Interestingly, DCA significantly attenuated 18S mRNA accumulation by 88% and 32% compared to coccidiosis and NE control birds, respectively (FIG. 14). These data suggest that DCA attenuates NE-induced inflammatory response and reduces the virulent activities of NE pathogens of C. perfringens and E. maxima.

### REFERENCES

Boyanova, L., Gergova, G., Spassova, Z., Koumanova, R., Yaneva, P., Mitov, I., Derejian, S., and Krastev, Z. (2004). Campylobacter infection in 682 bulgarian patients with acute enterocolitis, inflammatory bowel disease, and other chronic intestinal diseases. Diagn Microbiol Infect Dis 49, 71-74.
Buffie, C. G., Bucci, V., Stein, R. R., McKenney, P. T., Ling, L., Gobourne, A., No, D., Liu, H., Kinnebrew, M., Viale, A., et al. (2015). Precision microbiome reconstitution restores bile acid mediated resistance to Clostridium difficile. Nature 517, 205-208.
CDC (2014). Campylobacter general inflammation.
CDC (2018). Foodborne Diseases Active Surveillance Network (FoodNet) Fast (Centers for Disease Control and Prevention. https://wwwn.cdc.gov/foodnetfast/).
Gaucher, M. L., Quessy, S., Letellier, A., Arsenault, J., and Boulianne, M. (2015). Impact of a drug-free program on broiler chicken growth performances, gut health, Clostridium perfringens and Campylobacter jejuni occurrences at the farm level. Poult Sci 94, 1791-1801.
Geisinger, E., and Isberg, R. R. (2017). Interplay Between Antibiotic Resistance and Virulence During Disease Promoted by Multidrug-Resistant Bacteria. J Infect Dis 215, S9-S17.
Gradel, K. O., Nielsen, H. L., Schonheyder, H. C., Ejlertsen, T., Kristensen, B., and Nielsen, H. (2009). Increased short- and long-term risk of inflammatory bowel disease after salmonella or campylobacter gastroenteritis. Gastroenterology 137, 495-501.
Khan, A. U., Maryam, L., and Zarrilli, R. (2017). Structure, Genetics and Worldwide Spread of New Delhi Metallo-beta-lactamase (NDM): a threat to public health. BMC Microbiol 17, 101.
Lippert, E., Karrasch, T., Sun, X., Allard, B., Herfarth, H. H., Threadgill, D., and Jobin, C. (2009). Gnotobiotic IL-10; NF-kappaB mice develop rapid and severe colitis following Campylobacter jejuni infection. PloS one 4, e7413.
Mataki, C., Magnier, B. C., Houten, S. M., Annicotte, J. S., Argmann, C., Thomas, C., Overmars, H., Kulik, W., Metzger, D., Auwerx, J., et al. (2007). Compromised intestinal lipid absorption in mice with a liver-specific deficiency of liver receptor homolog 1. Mol Cell Biol 27, 8330-8339.
Newman, A., and Lambert, J. R. (1980). Campylobacter jejuni causing flare-up in inflammatory bowel disease. Lancet 2, 919.
Pham, N. T., Thongprachum, A., Tran, D. N., Nishimura, S., Shimizu-Onda, Y., Trinh, Q. D., Khamrin, P., Ukarapol, N., Kongsricharoern, T., Komine-Aizawa, S., et al. (2016). Antibiotic Resistance of Campylobacter jejuni and C. coli Isolated from Children with Diarrhea in Thailand and Japan. Jpn J Infect Dis 69, 77-79.
Ridlon, J. M., Kang, D. J., and Hylemon, P. B. (2006). Bile salt biotransformations by human intestinal bacteria. Journal of lipid research 47, 241-259.
Silverman, M. S., Davis, I., and Pillai, D. R. (2010). Success of self-administered home fecal transplantation for chronic Clostridium difficile infection. Clin Gastroenterol Hepatol 8, 471-473.
Speed, B., Kaldor, J., and Cavanagh, P. (1984). Guillain-Barre syndrome associated with Campylobacter jejuni enteritis. J Infect 8, 85-86.
Sun, X., Winglee, K., Gharaibeh, R. Z., Gauthier, J., He, Z., Tripathi, P., Avram, D., Bruner, S., Fodor, A., and Jobin, C. (2018). Microbiota-Derived Metabolic Factors Reduce Campylobacteriosis in Mice. Gastroenterology 154, 1751-1763 e1752.
Watkins, R. R., and Bonomo, R. A. (2016). Overview: Global and Local Impact of Antibiotic Resistance. Infect Dis Clin North Am 30, 313-322.
Wise, M. G., and Siragusa, G. R. (2007). Quantitative analysis of the intestinal bacterial community in one- to three-week-old commercially reared broiler chickens fed conventional or antibiotic-free vegetable-based diets. J Appl Microbiol 102, 1138-1149.

1. An animal feed composition comprising a bile acid or salt thereof and a protein component, carbohydrate component, or both a protein component and a carbohydrate component.

2. (canceled)

3. The animal feed composition of claim 1, wherein the bile acid comprises deoxycholic acid or a salt thereof.

4. The animal feed composition of claim 1, wherein the bile acid or a salt thereof is between 0.5 and 2 g/kg feed of the animal feed composition.

5. The animal feed composition of claim 1, wherein the animal feed composition is a poultry feed composition.

6. (canceled)

7. A method for, in an agricultural animal species, improving body weight gain, the accumulative feed conversion ratio, or preventing and/or reducing the growth of a pathogenic bacteria or a parasite in the intestinal tract of the agricultural animal species comprising administering to the agricultural animal species an effective amount of a composition comprising a bile acid or a salt thereof.

8. The method of claim 7, wherein the agricultural animal species is a poultry species.

9. The method of claim 7, wherein the bacteria or the parasite is selected from the group consisting of Campylobacter jejuni, Eimeria maxima, and Clostridium perfringens.

10. The method of claim 7, wherein the bile acid comprises deoxycholic acid or a salt thereof.

11. The method of claim 7, wherein the composition comprises a bile acid or salt thereof and a protein component, carbohydrate component, or both a protein component and a carbohydrate component.

12. (canceled)

13. A bile acid modulated intestinal digesta or a bile acid modulated anaerobe intestinal digesta.

14. The intestinal digesta of claim 13, wherein the bile acid comprises deoxycholic acid (DCA) or a salt thereof.

15. (canceled)

16. An animal feed composition comprising the intestinal digesta of claim 13 and a protein component, carbohydrate component, or both a protein component and a carbohydrate component.

17. A method of making the bile acid modulated intestinal digesta of claim 13 comprising:
administering a bile acid or a salt thereof to an agricultural animal species, and
isolating an intestinal digesta from the agricultural animal species.

18. The method of claim 17, further comprising culturing the intestinal digesta under anaerobic conditions.

19. The method of claim 17, wherein the agricultural animal species is a poultry species.

20. The method of claim 17, wherein the bile acid comprises deoxycholic acid (DCA) or a salt thereof.

21. (canceled)

22. A method for preventing and/or reducing the growth of a pathogenic bacteria or a parasite in the intestinal tract of an agricultural animal species comprising administering to the agricultural animal species an effective amount of the composition of claim 13.

23. The method of claim 22, wherein the bacteria or the parasite is selected from the group consisting of Campylobacter jejuni, Eimeria maxima, and Clostridium perfringens.

24. The method of claim 22, wherein the agricultural animal species is a poultry species.

25. The method of claim 22, wherein the composition comprises a deoxycholic acid (DCA) modulated anaerobe cecal digesta.
